(19) United States
(12) Patent Application Publication
(10) Pub. No.: US 2009/0222965 A1
(43) Pub. Date: Sep. 10, 2009

(54) SAFETY GLASSES FOR CLEAN ROOMS AND METHOD FOR SECURING SAME

(76) Inventor: Frederic Behar, LE VESINET (FR)

Correspondence Address: BLAKELY SOKOLOFF TAYLOR & ZAFMAN LLP, 1279 OAKMEAD PARKWAY, SUNNYVALE, CA, 94085-4040, US

(21) Appl. No.: 12/398,108

(22) Filed: Mar. 4, 2009

(30) Foreign Application Priority Data
Mar. 5, 2008 (FR) 0851437

(51) Int. Cl. A61F 9/00

(52) U.S. Cl. 2/10; 2/11

(57) ABSTRACT

Safety glasses for clean room, comprising two lateral fastening buckles enabling securing for the user of said glasses by manipulating only said fastening buckles and without having to manipulate the rest of said glasses.

The invention concerns the field of clothing, work clothing, equipment and accessories for clean rooms, i.e. for rooms with controlled atmospheres.

More particularly, the invention relates to safety glasses for clean rooms.

It also relates to a method for securing these glasses on a jumpsuit, a hood or any other type of cap adapted for clean rooms.

Traditionally, people intended to work in clean rooms are provided with safety glasses which comprise, for example, a rim going back toward the user's face in order to protect the user's eyes as well as possible.

These glasses are also used to limit the emission of particles, such as scales, eyelashes, etc, in clean rooms.

Glasses of this type may, for example, be spectacles-type glasses or glasses with elastic straps.

In the latter case, when an operator puts the glasses on, he first places the glasses in front of his face, at eye level, then holds the frame and/or the brow rest with one hand and pulls the strap toward the back of the head with the other hand.

There are, on average, five contacts between the operator's hands and the glasses.

This is a drawback, especially when the operator then has to enter a clean room and, a fortiori, a sterile room.

Indeed, the atmosphere of a clean room is monitored, the concentration and the size of the particles are constantly checked. If the concentration of particles is too high and/or if the size of these particles becomes too large, these particles can contaminate work stations and severely damage the quality of the operator's work.

Furthermore, in a sterile room, the operator needs to introduce as few germs as possible inside the room when he enters it.

The more the outer part of the clothing and accessories put on and worn by the operator comes into contact with another object (clothing and fingers, in particular), the greater the risk on one hand of creating particles which will come loose and pollute the room's atmosphere, and on the other hand of depositing germs which can contaminate the room's atmosphere.

One aim of the invention is therefore to propose glasses which can be easily put on while avoiding contact.

Another aim of the invention is to provide a method for securing these glasses on a jumpsuit, a hood or any other type of cap adapted for clean rooms, minimizing contact.

To these aims, the invention provides safety glasses for a clean room comprising two lateral fastening buckles, wherein each of the fastening buckles is a flat strip, comprising an inner surface by which the user manipulates said buckles during securing of the glasses, and an outer surface which is designed to remain out of contact with the user, whereby enabling the user to secure said glasses by manipulating only said fastening buckles without having to manipulate the rest of said glasses.

Other preferred, but non-limiting features of these safety glasses are:
the fastening buckles are connected respectively to two lateral parts of said glasses or, if applicable, to their frame;
the two fastening buckles are made from bands whereof the width is from approximately 1 cm to approximately 5 cm;
the glasses have lateral slots through which the bands that form the fastening buckles are slipped;
the fastening buckles are folded over themselves by snap fastener or by stitching;
the fastening buckles have snap fasteners;
the glasses are presented in a sterile, individual packaging.
   The invention also provides a method for securing safety glasses according to the invention on a jumpsuit comprising a head portion, on a hood, or on any other type of cap adapted for clean rooms, the head portion, the hood or the cap adapted for clean rooms having lateral tightening straps arranged on the head portion and at the temples, the method comprising:
slipping fingers of a first right or left hand through the respective fastening buckle, touching only the inner surface of the fastening buckle;
keeping the fingers of the first hand through the fastening buckle, and slipping fingers of a second left or right hand through the other respective fastening buckle, touching only the inner surface of the fastening buckle.

Other preferred, but non-limiting characteristics of this method are:
the method also comprises:
   grasping with the fingers of each hand, these fingers still being slipped through the fastening buckles, the respective tightening strap of the jumpsuit;
   pulling the lateral tightening straps while freeing the fingers from the fastening buckle;
   tying the two lateral tightening straps together behind the head with a knot;
pulling the lateral tightening straps while freeing the fingers from the fastening buckles is done by first pulling outward, then backward;
pulling the lateral tightening straps while freeing the fingers from the fastening buckles is done by also pulling upward while pulling backward;
the head portion, the hood or the cap adapted for clean rooms has snap fasteners; and in that the method also comprises:
   pressing the snap fasteners of the fastening buckles with the snap fasteners of the top part, of the hood, of the cap adapted for clean rooms, touching only the inner surface of the fastening buckles;
   freeing the fingers from the buckles; and
   tying the two lateral tightening straps together by making a knot behind the head;
once the fingers of one hand are slipped into a fastening buckle, the fingers are removed;
before slipping the fingers of the first hand through the respective fastening buckle, it comprises freeing the glasses from a packaging while only holding the glasses through the packaging.

One advantage using the safety glasses and the method of securing the glasses is that the number of contacts necessary for placement and securing of the safety glasses is decreased.

Other features, aims and advantages of the present invention will appear upon reading the detailed description which follows, with reference to the appended drawings, provided as non-limiting examples and in which:

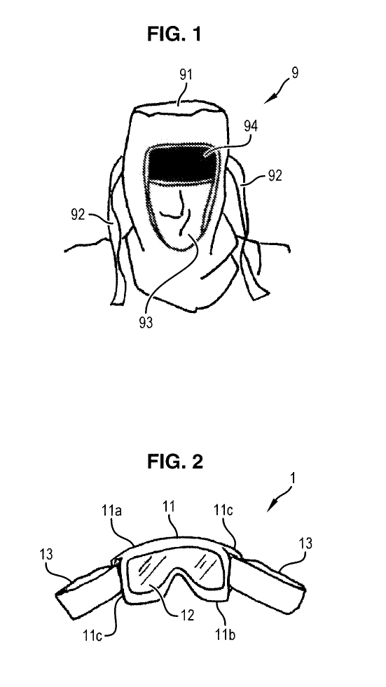

FIG. 1 is an illustration of a top part of a traditional jumpsuit used by an operator in a clean room;

FIG. 2 is a schematic illustration of safety glasses according to an embodiment of the invention;

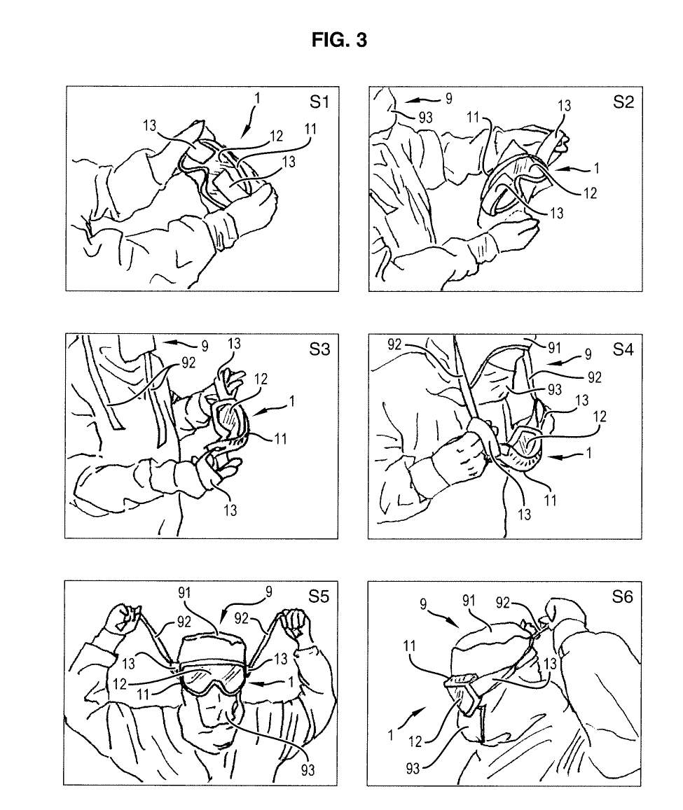

FIG. 3 sequentially illustrates the steps of a method for securing the safety glasses.

In reference to FIG. 1, hereinafter is described a top portion 9 of a traditional jumpsuit used by an operator in a clean room.

This top portion 9 comprises a head portion 91 surrounding the head of the operator and two lateral tightening straps 92 placed on either side of the head portion 91 substantially at the operator's temples once the jumpsuit is put on.

In one variation, the head portion 91 covers the operator's head so as to form a defined space 94 leaving an uncovered zone at the level of the operator's eyes.

In another variation, the head portion 91 covers the operator's head by going around his face and the top portion 9 of the jumpsuit also comprises a mask portion 93.

The mask portion 93 covers the bottom of the operator's face up to below the eyes. The mask portion 93 is attached to the head portion 91 by stitching so that only a defined space 94 is left making it possible to leave the operator's eyes uncovered.

In normal traditional use, the lateral tightening straps 92 on either side of the head portion 91 are used to keep said portion in place on the operator's head so that it does not bother the operator, for example by sliding. To do this, the operator pulls the lateral tightening straps toward the back of his head and ties them in a knot.

In variation, the head portion 91 has snap fasteners (for example, three per side) on each side, at the operator's temple and between the edge of the head portion and the lateral tightening straps 92. These snap fasteners are arranged so as to be substantially aligned along a horizontal direction.

Lastly, some jumpsuits do not comprise a top portion 9. In these cases, the operator uses a hood, or any type of cap adapted for clean rooms, having the same features as the top portion 91 described above, and which therefore will not be described any further.

With reference to FIG. 2, safety glasses 1 according to the invention are described.

These safety glasses 1 comprise a brow rest 12 and two fastening buckles 13.

Hereafter, brow rest 12 designates the tinted or untinted transparent part of the glasses, through which the operator can see.

In variation, the safety glasses also comprise a frame 11.

The glasses and/or, if applicable, the frame 11 are substantially B-shaped generally with a more or less straight upper portion 11a (ascender of the B), two lateral portions 11c (upper and lower hair lines of the B) and a curved lower portion 11b (spine of the B).

If applicable, the frame 11 is, for example, made in thermoplastic polyvinyl chloride (PVC).

The brow rest 12 covers the surface of the B. The brow rest 12 is made, for example, in poly methyl methacrylate or in polycarbonate.

The two fastening buckles 13 are made from relatively wide flat bands whereof the width is from approximately 1 cm to approximately 5 cm.

The flat bands are made in synthetic materials, for example in continuous polyester yarn or any other non-contaminant material, i.e. which does not drop particles.

The two fastening buckles 13 are respectively connected to the two lateral portions 11c of the safety glasses 1. The connection can be done in various ways.

The fastening buckles 13, once attached to the glasses, and/or if applicable to the frame 11, have an outer surface designed to remain out of contact with the operator and an inner surface by which the operator manipulates said fastening buckles 13 while securing of the glasses 1.

In one variation, the glasses and/or, if applicable, the frame 11, have, at their two lateral portions 11c, elongated lateral slots. The length of these slots is substantially equal to the width of the fastening buckles 13. Thus, the bands forming the fastening buckles 13 are inserted through these slots, then folded over themselves by snap fastener, stitching or any other adapted means.

In another variation, the fastening buckles 13 are formed from flat bands which can be folded over themselves, and the glasses, and/or if applicable the frame 11, are cast onto the fastening buckles 13, such that the latter are closed.

In variation, the fastening buckles 13 have snap fasteners (for example, three) on their outer surface. These snap fasteners are arranged, substantially aligned, on the length of the fastening buckles 13. This variation is, optionally but non-restrictively, used with a head portion 91 or a hood (or any other type of cap adapted for clean rooms) provided with snap fasteners as described above. The snap fasteners of the fastening buckles 13 have shapes which complementarily match shapes of the snap fasteners of the head portion or the hood (or any other type of cap adapted for clean rooms).

The glasses and/or, if applicable, the frame 11 have extensions substantially perpendicular to the average plane of the B. These extensions allow the glasses to substantially fit the shape of the operator's face at eye level.

One advantage of these safety glasses 1 is that the material of the fastening buckles 13 is resistant to cleaning and sterilization methods, contrary to the materials of the fastening systems currently used, such as elastic.

With reference to FIG. 3, is now described a first exemplary embodiment of the method for securing these safety glasses 1.

The method for securing the safety glasses 1 according to the first exemplary embodiment includes the following steps.

Step 1 (S1). In order to meet the requirements for work in a clean room, the safety glasses 1 are presented in a sterile individual packaging. Therefore, the operator holds the glasses 1 through this packaging in step 1.

The operator then opens the packaging on one of the sides corresponding to a side where a fastening buckle 13 is, and frees the latter part while being careful not to touch the outer surface, designed to remain out of contact with the operator, of the fastening buckle 13.

Step 2 (S2). He then slips the fingers of a first right or left hand through the respective fastening buckle 13, still without touching the outer surface of the fastening buckle; he thus only touches the inner surface.

Step 3 (S3). While keeping the fingers of his first hand through the fastening buckle 13, he then slips the fingers of his second left or right hand through the other respective fastening buckle 13, without touching the outer surface thereof; he thus only touches the inner surface.

In order to stabilize the glasses 1, the operator can move the fingers of his first hand away so that the glasses 1 do not oscillate too much.

Step 4 (S4). With the fingers of each hand, the operator grasps the respective lateral tightening straps 92 of the head portion 91 of the jumpsuit.

Step 5 (S5). The operator pulls on the lateral tightening straps 92 while freeing his fingers from the fastening buckles 13 first outward then backward and eventually upward.

Step 6 (S6). The operator lastly ties the two lateral tightening straps 92 together, making a knot behind his head.

Below is described a second exemplary embodiment of the method according to the invention. This embodiment is used with the variations of glasses and jumpsuits or hoods (or any other type of cap adapted for clean rooms) provided with snap fasteners.

This embodiment comprises the same steps 1, 2 and 3 as the first exemplary embodiment. It also comprises the following steps.

Step 4′. With the fingers of each hand, the operator pushes the snap fasteners of the fastening buckles 13 with the snap fasteners of the top portion 91 or of the hood (or of any other type of cap adapted for clean rooms), touching only the inner surface of the fastening buckles 13, and frees his fingers from the buckles.

Step 5′. The operator lastly ties the two lateral tightening straps 92 together, making a knot behind his head.

The safety glasses 1 are then secured and well-positioned, as well as the top portion 9 of the jumpsuit.

A method of this type also has a certain advantage in the pharmaceutical or biological industries, where clean rooms must remain sterile. Indeed, the more one decreases the number of contacts on the surfaces exposed to the room's atmosphere, the more the risk of introducing microorganisms into the atmosphere of the room decreases.

It is, however, also advantageous to use it in clean rooms whereof the atmosphere is very controlled and for which one wishes to have a very low concentration of particles suspended in the air.

Lastly, one advantage of this method lies in the fact that, regardless of the clothing protocol used, contaminations or soiling of the surfaces in contact with the atmosphere of the clean room are limited. Indeed, according to the protocol, the operator may or may not wear gloves while putting on his glasses. If he does not wear gloves, he must then touch the glasses. With the method according to the invention, he will only touch the inner surfaces of the fastening buckles, which will not be in contact with the atmosphere of the clean room.

The present invention is not limited to the embodiments described above, but extends to all embodiments in line with its spirit.

1. Safety glasses for clean room comprising two lateral fastening buckles, wherein each of the fastening buckles is a flat strip, comprising an inner surface by which the user manipulates said buckles during securing of the glasses and an outer surface which is designed to remain out of contact with the user, whereby enabling the user to secure said glasses by manipulating only said fastening buckles without having to manipulate the rest of said glasses.

2. The glasses according to claim 1, wherein the fastening buckles are connected to two lateral portions of said glasses or, if applicable, of their frame, respectively.

3. The glasses according to claim 2, wherein the two fastening buckles are made from bands whereof the width is from approximately 1 cm to approximately 5 cm.

4. The glasses according to claim 2 wherein they have lateral slots through which the bands forming the fastening buckles are slipped.

5. The glasses according to claim 1, wherein the fastening buckles are folded over themselves by snap fastener or by stitching.

6. The glasses according to claim 1, wherein the fastening straps have snap fasteners.

7. The glasses according to claim 1, wherein they are presented in a sterile individual packaging.

8. A method for securing safety glasses on a jumpsuit comprising a head portion, on a hood, or on any other type of cap adapted for clean rooms, the head portion, the hood or the cap adapted for clean rooms having lateral tightening straps arranged on the head portion and at the temples, the safety glasses comprising two lateral fastening buckles, each of the fastening buckles being a flat strip, comprising an inner surface by which the user manipulates said buckles during securing of the glasses and an outer surface which is designed to remain out of contact with the user, whereby enabling the user to secure said glasses by manipulating only said fastening buckles without having to manipulate the rest of said glasses, the method comprising:
slipping the fingers of a first right or left hand through the respective fastening buckle, touching only the inner surface of the fastening buckle;
keeping the fingers of the first hand through the fastening buckle, and slipping the fingers of a second left or right hand through the other respective fastening buckle, touching only the inner surface of the fastening buckle.

9. The method according to claim 8, wherein it also comprises:
grasping with the fingers of each hand, these fingers still being slipped through the fastening buckles, the respective tightening strap of the jumpsuit;
pulling the lateral tightening straps while freeing the fingers from the fastening buckle;
tying the two lateral tightening straps together behind the head with a knot.

10. The method according to claim 9, wherein pulling the lateral tightening straps while freeing the fingers from the fastening buckles is done by first pulling outward, then backward.

11. The method according to claim 10, wherein pulling the lateral tightening straps while freeing the fingers from the fastening buckles is also done by pulling upward while pulling backward.

12. The method according to claim 8 for safety glasses comprising two lateral fastening buckles, each of the fastening buckles being a flat strip, comprising an inner surface by which the user manipulates said buckles during securing of the glasses and an outer surface which is designed to remain out of contact with the user, whereby enabling the user to secure said glasses by manipulating only said fastening buckles without having to manipulate the rest of said glasses, the fastening snaps having snap fasteners, wherein the head portion, the hood or the cap adapted for clean rooms has snap fasteners; and wherein the method also comprises:
pressing the snap fasteners of the fastening buckles with the snap fasteners of the top portion, of the hood, the cap adapted for clean rooms, touching only the inner surface of the fastening buckles;
freeing the fingers from the buckles; and
tying the two lateral tightening straps together by making a knot behind the head.

13. The method according to claim 8, wherein once the fingers of one hand are slipped into a fastening buckle, the fingers are moved away.

14. The method according to claim 8, wherein before slipping the fingers of the first hand through the respective fastening buckle, the method comprises freeing the glasses from a packaging while only holding the glasses through the packaging.
